(19) United States
(12) Patent Application Publication
(10) Pub. No.: US 2013/0011857 A1
(43) Pub. Date: Jan. 10, 2013

(54) COMPLEMENT FACTOR H FOR OXIDATIVE STRESS DISEASE CONDITIONS

(75) Inventors: Christoph Binder, Wien (AT); David Weismann, Wien (AT)

(73) Assignee: CEMM FORSCHUNGSZENTRUM FUR MOLEKULARE MEDIZIN GMBH, Wien (AT)

(21) Appl. No.: 13/578,582

(22) PCT Filed: Feb. 4, 2011
(86) PCT No.: PCT/EP2011/051652

§ 371 Date: Aug. 30, 2012

Related U.S. Application Data
(60) Provisional application No. 61/303,796, filed on Feb. 12, 2010.

(30) Foreign Application Priority Data
Feb. 12, 2010 (EP) 10153457.6

(51) Int. Cl. G01N 33/566

(52) U.S. Cl. 435/7.4

(57) ABSTRACT

The invention relates to complement Factor H for use in the prevention and treatment of oxidative stress disease conditions in a patient, the use of Factor H in the preparation of a pharmaceutical preparation, and methods of determining the specific binding of Factor H to MDA and/or MAA in a sample.

The invention refers to complement Factor H for pharmaceutical use.

### BACKGROUND

Microorganisms or toxins that successfully enter an organism will encounter the cells and mechanisms of the innate immune system. The innate response is usually triggered when microbes are identified by pattern recognition receptors, which recognize components that are conserved among broad groups of microorganisms, or when damaged, injured or stressed cells send out alarm signals, so called “danger signals”, many of which are recognized by the same receptors as those that recognize pathogens. Innate immune defenses are non-specific. This system does not confer long-lasting immunity against a pathogen in the sense of adaptive memory responses. The innate immune system is the dominant system of host defense in most organisms.

Inflammation is one of the first responses of the immune system to infection or any other insult, e.g. mechanical. The symptoms of inflammation are redness and swelling, which are caused by increased blood flow into a tissue. Inflammation is promoted by eicosanoids and cytokines, which are released by injured or infected cells. Eicosanoids include prostaglandins that produce fever and the dilation of blood vessels associated with inflammation, and leukotrienes that attract leukocytes. Common cytokines include interleukins that are responsible for communication between white blood cells; chemokines that promote chemotaxis; and interferons that have anti-viral effects, such as shutting down protein synthesis in the host cell. Growth factors and cytotoxic factors may also be released. These cytokines and other chemicals recruit immune cells to the site of infection and promote healing of any damaged tissue following the removal of pathogens.

The complement system is a biochemical cascade that attacks the surfaces of foreign cells. It contains over 20 different proteins and is named for its ability to “complement” the killing of pathogens by antibodies. Complement is the major humoral component of the innate immune response. In humans, this response is activated by complement binding to antibodies that have attached to these microbes or the binding of complement proteins to carbohydrates on the surfaces of microbes. This recognition signal triggers a rapid killing response. The speed of the response is a result of signal amplification that occurs following sequential proteolytic activation of complement molecules, which are also proteases. After complement proteins initially bind to the microbe, they activate their protease activity, which in turn activates other complement proteases, and so on. This produces a catalytic cascade that amplifies the initial signal by controlled positive feedback. The cascade results in the production of peptides that attract immune cells, increase vascular permeability, and opsonize the surface of a pathogen, marking it for destruction. This deposition of complement can also kill cells directly by disrupting their plasma membrane.

Natural antibodies (NAbs) play an important immunobiological role in the natural defense mechanism. NAbs spontaneously arise without prior infection or immune exposure. In mice, they are predominantly derived from B-1 cells. Natural antibodies exhibit a remarkably conserved repertoire, which has been suggested to represent a primitive layer of the immune system as a product of natural selection. NAbs are typically regarded as “polyreactive” in that they bind to a number of self or foreign antigens. This pattern of broad reactivity of a preformed pool of antibodies is required for the rapid and immediate recognition and protection against invading pathogens. On the other hand, natural antibodies may also play a role in the recognition and removal of senescent cells, cell debris, and other (neo-)self-antigens and thereby possess another so called “house-keeping” function in neutralizing and removing body waste and protecting from autoimmunity. There has been evidence of pathways in which NAbs may contribute in the elimination of self-antigens exposed during stress, tissue damage, or even conventional cell turnover.

Atherosclerosis is a disease of the vascular wall that leads to myocardial infarction, heart failure, peripheral vascular disease, and stroke. Although multiple risk factors have been identified that contribute variably to lesion formation, the growth of the atherosclerotic lesion is both initiated and sustained by increased levels of LDL and low and/or dysfunctional HDL. In the past decade, inflammatory processes have been identified as equally important factor contributing to atherosclerotic lesion formation. Atherosclerosis develops over decades and is believed to progress from intimal thickening to ever more complex lesions involving the accumulation of cells derived from the circulation, proliferation of inherent vascular wall cells, and synthesis of extracellular matrix, and lipid accumulation, both extracellular bound to matrix and intracellular, within macrophage foam cells. Macrophage cholesteryl ester formation is believed to be attributable in large part to enhanced and unregulated uptake of oxidized, aggregated, and variously otherwise modified LDLs and possibly other lipoproteins and disturbed cellular responses that are unable to mediate the export of the accumulated cholesterol load. As the lesions progress, many of the lipid-filled cells undergo apoptosis but are not sufficiently cleared, leading to an abnormal accumulation of apoptotic cells in the lesion. Under these conditions, apoptotic cells may undergo secondary necrosis, yielding the acellular gruel characteristic of the advanced atherosclerotic plaques. Smooth muscle cell proliferation and secretion of a thick collagen cap may stabilize the lesion, but eventually vulnerable areas of the plaque erode or rupture, leading to thrombosis, ischemia, and clinical events or even death.

Oxidation-specific epitopes are a class of pathogen-associated molecular patterns (PAMPs) that are recognized by natural antibodies and other innate and adaptive immune receptors. Physiological and pathological stress can lead to the generation of oxidation-specific epitopes, which are considered altered self or neoself-antigens on membranes of lipoproteins as well as (apoptotic or necrotic) cells or cellular debris, which are subsequently recognized by natural antibodies, scavenger receptors, and other innate effector proteins via these motifs. In many, if not all, cases, molecular mimicry exists between oxidation-specific epitopes of self-antigens and epitopes of microbes, which represent the “conserved” patterns on various pathogens to which NAbs bind, termed “pathogen-associated molecular patterns” (PAMPs) (Shaw P. X. et al. 2000. J. Clin. Invest. 105: 1731-1740). Thus, as a result of oxidative stress, oxidation-specific epitopes constitute one category of “altered self,” which represents “danger signals” (e.g. PAMPs) that are recognized and defended against by multiple arcs of innate immunity.

Typically, peroxidation of the abundant phospholipid phosphatidylcholine is initiated at the oxidation prone sn-2 polyunsaturated fatty acid. Decomposition of the oxidized fatty acid generates a wide spectrum of reactive molecular species, such as malondialdehyde (MDA) with its condensation products and 4-hydroxynonenal (HNN), as well as the “core aldehyde” of the residual oxidized phospholipid (OxPL) backbone, yielding 1-palmitoyl-2-(5-oxovaleroyl)-sn-glycero-3-phosphocholine (POVPC), which contains the phosphorylcholine (PC) head group. These reactive aldehydes can modify autologous molecules, including both the protein moiety of LDL, apolipoprotein B (apoB), and other lipid molecules, such as amine-containing phospholipids (e.g., phosphatidylserine). Thus, altered lipids as well as oxidized lipid-protein adducts are formed, yielding, for example, MDA-modified lysines on proteins as well as aminophospholipids, as well as OxPLs and OxPLprotein/lipid adducts.

MDA is also present in atherosclerotic lesions. MDA-modified (adducted) proteins, including MDA-modified LDL, are present in atherosclerotic human vascular tissue. Acetaldehyde (AA) is the major metabolic product of ethanol oxidation. Both MDA and AA are highly reactive aldehydes and will combine with proteins to produce an antigenically distinct, immunogenic protein adduct, termed the Malonacetaldehyde (MAA) adduct. Proteins modified in the presence of high concentrations of MDA can also produce MAA-modified proteins.

Chang M. K. et al. (1999 Proc. Natl. Acad. Sci. USA. 96: 6353-6358.) demonstrated that a number of the different oxidation-specific IgM monoclonal antibodies that bound OxLDL, such as EO6, and EO14, which binds to MDA-LDL, bound to cell surface determinants on apoptotic cells but not viable cells. Furthermore, each of these antibodies could inhibit the uptake of apoptotic cells by macrophages in an additive manner. These oxidation-specific neoepitopes are evidently PAMPs representing “eat me” signals to innate immunity, as manifested by macrophage scavenger receptors and natural antibodies. It is likely that there are many other such neoepitopes generated as a result of stress-induced alterations in native structures. These epitopes can be generated by adduct formation between reactive lipid moieties and proteins or other lipids, generating entirely novel structures, such as MDA-lysine adducts on LDL (Binder C. J. et al. 2005 J. Lipid Res. 46: 1353-1363).

C-reactive protein (CRP) was found to bind to an oxidation-specific epitope, namely phosphocholine (PC) of oxidized but not unoxidized phospholipids. CRP is an acute phase protein that has been widely studied as a sensitive marker of inflammation and is a powerful prognosticator with respect to cardiovascular disease, as discussed by Chait A. et al. (2005. J. Lipid Res. 46: 389-403.). CRP was originally recognized for its ability to bind to the cell wall of S. pneumoniae, specifically to the PC moiety that is covalently linked to teichoic or lipoteichoic acid.

Complement Factor H is a member of the regulators of complement activation family and is a complement control protein. Factor H is a big plasma glycoprotein of 155 kDa that circulates in human plasma at a concentration of 500-800 micrograms per milliliter. It is the major inhibitor of the alternative pathway of complement, ensuring that the complement system is directed towards pathogens and does not damage host tissue. Factor H regulates complement activation on self cells by possessing both cofactor activity for the Factor I mediated C3b cleavage, and decay accelerating activity against the alternative pathway C3 convertase, C3bBb. Factor H protects self cells from complement activation but not bacteria/viruses, in that it binds to glycosaminoglycans (GAGs) that are present on host cells but not pathogen cell surfaces. Factor H deficiency correlates with various diseases, including the kidney disease membranoproliferative glomerulonephritis as well as age related macular degeneration and cardiovascular disease.

Since mutations or single nucleotide polymorphisms (SNPs) in Factor H often result in pathologies, Factor H is also considered a risk factor for complement mediated diseases, e.g. Age-Related Macular Degeneration (AMD). It was discovered that about 35% of individuals carry an at-risk SNP in one or both copies of their Factor H gene. Homozygous individuals have an up to sevenfold increased chance of developing age-related macular degeneration, while heterozygotes have a two-to-threefold increased likelihood of developing the disease. This SNP, located in complement control protein (CCP) module 7 of Factor H, indicates a causal relationship between the SNP and disease. The variant in the Factor H gene is a polymorphism which results in the substitution of T to C nucleotide at position 1277 in exon 9; one form of the Factor H, also called isoform, has the amino acid histidine at position 402 and the other variant has a tyrosin (Y402). It is the histidine form that associates with AMD and other inflammatory diseases.

WO2008/090332A2 describes the Factor H polymorphisms in the diagnosis and therapy of inflammatory diseases.

WO2008/113589 A1 describes methods for the production of therapeutic Factor H preparations from human plasma, for use in a substitution therapy.

Huang et al. (2008 The Journal of Immunology 181: 8068-8076) disclose a fragment of complement receptor 2 (CR2), a receptor that targets C3 activation products, which is fused to Factor H to inhibit the alternative pathway of complement. While endogenous serum Factor H would fail to provide protection against intestine ischemia/reperfusion injury, the fusion protein was found to effectively protect from local (intestine) and remote (lung) injury.

WO2006/062716 describes a composition comprising Factor H for the treatment of age-related macular degeneration (AMD).

WO2007/056227 describes the use of a complement inhibitor for the treatment of ocular diseases, e.g. AMD, diabetic retinopathy, and ocular angiogenesis. Amongst others Factor H is mentioned as complement inhibitor.

US2007/0020647 describes the use of a recombinant Factor H for the treatment of AMD.

WO2008/120215 discloses a temporary surgical implant with a biomimetic coating comprising Factor H.

U.S. Pat. No. 4,883,784 relates to the use of Factor H for the therapy of autoimmune diseases, such as systemic lupus erythematosus, rheumatoid arthritis and glomerulonephritis.

U.S. Pat. No. 6,248,365 discloses the use of Factor H for the prophylaxis and therapy of chronic inflammatory intestinal disorders, inflammatory skin disorders and purpura.

US2008/0318841 relates to the use of Factor H for the treatment of Uremic Haemolytic Syndrome (UHS).

### SUMMARY

There is a need to better understand the natural defense mechanism in detail and to cure diseases resulting from an imbalance of the innate immune system. It is, thus, an object of the invention to provide measures to support the natural defense mechanism, specifically directed against oxidation-specific epitopes, thus, enabling the treatment of disease conditions associated with oxidative stress.

The object is solved by the subject matter of the claims. Thus, the invention refers to complement Factor H, including Factor H related proteins for use in the prevention and treatment of oxidative stress disease conditions in a patient.

Specifically the Factor H is provided in a purified preparation with an increased MDA binding capacity when compared to a preparation purified from normal human plasma or normal human serum or the purified 402H Factor H isoform. Normal human plasma or serum is typically produced on a large scale, e.g. from pooled sources with a pool size of at least 100, specifically at least 1000 plasma or blood donations. The binding capacity is preferably determined in the purified preparation and compared to the purified preparation with about the same degree of purification. An increased binding capacity is particularly determined, if the increase is by at least 25%, specifically at least 50%, 75%, 100%, or even more, possibly up to 250%.

Preferably the Factor H preparation according to the invention is enriched in Factor H variants with high MDA binding capacity , such as Factor H 402Y, and optionally depleted of variants with reduced MDA binding capacity, such as the 402H Factor H variant.

Specifically the invention refers to the oxidative stress, which is associated with cardiovascular disease, the metabolic syndrome and obesity, autoimmune diseases, including rheumatoid arthritis, multiple sclerosis, cancer and conditions caused by cancer treatment, age related macular degeneration, Alzheimer's disease, brain senescence, alcoholic liver disease, ischemic reperfusion injury, diabetic nephropathy, nephritis, acute lung injury, and infectious diseases, or any inflammatory conditions associated or caused therewith.

According to the invention the use of Factor H is preferably indicated when the patient suffers from a disease condition, which is associated with an increased level of at least one of an oxidative stress marker selected from the group consisting of oxidized lipoproteins, oxidized lipids and/or proteins, circulating microparticles, necrotic or apoptotic cells, cellular debris, and complexes of such oxidative stress marker with endogenous Factor H and/ or specific antibodies or an increased level of thiobarbituric acid reacting substances (TBARS).

Preferably this level is determined by an immunoassay specifically detecting bound endogenous malondialdehyde (MDA) and/or malondialdehyde-acetaldehyde (MAA) adducted proteins to Factor H and/or to MDA/MAA specific antibodies, in a sample of tissue or body fluid, such as blood, plasma or serum.

According to the invention, Factor H preferably inhibits MDA and/or MAA function, or the effects mediated by MDA and/or MAA.

Preferably the patient treated with Factor H according to the invention has an increased risk of disease conditions associated with low or dysfunctional levels of endogenous Factor H, e.g. characterized by the serum MDA binding capacity that is at least 25% less than normal human serum or normal human plasma, and specifically associated with or bearing the the 402H Factor H variant.

Preferably this increased risk is determined by an immunoassay specifically detecting binding of endogenous Factor H to MDA and/or MAA in a sample of tissue or body fluid, such as blood, plasma or serum.

According to the invention, the preferred Factor H dose is ranging between 5 and 100 IU/kg, preferably between 20 and 80 IU/kg, most preferred is a dose resulting in the level of Factor H in the normal range.

Factor H according to the invention is preferably provided in a preparation, having a specific MDA or MAA binding activity of at least 0.001 g MAA-modified protein per gram Factor H, as determined in a saturation assay. A specific activity is particularly preferred, which is within the range of activity of the physiological normal Factor H. In some cases, however, Factor H fragments or derivatives may be provided having an increased specific activity, such as molecules bearing only those peptide sequences relevant for MDA binding.

The Factor H according to the invention is preferably provided in a preparation for parenteral, intranasal, intrabronchial, intraocular or topical use.

In a particularly preferred embodiment of the invention the Factor H is devoid of complement receptor 2.

The preferred Factor H according to the invention is provided in a preparation that essentially consists of Factor H.

Preferably the Factor H according to the invention is a recombinant protein, fragment or variant thereof, containing at least domain 7 and/or domain 20, peptides derived therefrom or their homologues, specifically with respect to Factor H related protein. Respective homologues preferably have at least 70% sequence identity, preferably at least 80%, 90% or at least 95% sequence identity.

According to another preferred embodiment of the invention the Factor H, including Factor H related proteins, is provided in a preparation purified from human blood, plasma or serum, optionally a blood plasma pool. Specifically Factor H according to the invention is purified from a pool of human blood plasma, e.g. on a large scale, wherein the individual donor and/or the pool is selected for an increased MDA binding capacity, thereby significantly reducing the risk of undesired Factor H variants. Preferably the Factor H is separated from impurities containing MDA or MAA epitopes by purification through affinity chromatography.

According to a specific aspect of the invention there is provided a method of purifying a blood or plasma preparation by MDA or MAA affinity chromatography.

Preferably this preparation contains complement Factor H.

According to another aspect of the invention the Factor H is used in the preparation of a pharmaceutical preparation for the prevention or treatment of oxidative stress disease conditions in a patient.

According to a further aspect the invention refers to a method of determining the specific binding of Factor H to MDA and/or MAA in a sample comprising:

providing a sample of body tissue or fluid,

providing a reactand, which is either Factor H or epitopes selected from MDA and/or MAA epitopes, which reactand is optionally bound to a solid surface,

incubating said reactand with said sample, and

determining the reaction products of said sample with said reactand.

Depending on which of Factor H or the epitopes are determined in said sample the reactand comprises the respective binder, either in the soluble state or immobilized on a carrier. The preferred method according to the invention relates to the determination of endogenous Factor H, which binds to the MDA and/or MAA epitopes thereby forming reaction products, which endogenous Factor H is a prognostic factor of oxidative stress disease.

Other features and advantages of the present invention will become apparent from the detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

FIG. 1: Factor H binds specifically to MDA-modified proteins

Biotin-labeled MDA-LDL was mixed with indicated concentrations of unlabeled native LDL, MDA-LDL, MAA-LDL and OxLDL. The mixture was incubated with coated complement Factor H and the amount of bound biotin-labeled MDA-LDL was determined with AP-conjugated neutravidin. Each point is the mean of triplicate determinations, expressed as a ratio of biotin-MDA-LDL binding to complement factor H in the presence of competitor to the binding in the absence of competitor (B/B0).

FIG. 2: Factor H interacts with MDA via SCR7 and SCR20

Chemiluminescent immunoassay for the binding of full length complement factor H and recombinantly expressed fH-deletion mutants to coated BSA and MAA-BSA, respectively. Bound Factor H was detected with goat anti-human-Factor H antiserum. AP (alkaline phosphatase)-conjugated anti-goat-IgG was used as secondary antibody. Data are the mean of triplicate determinations.

FIG. 3: Factor H and MDA are present in AMD

Immunohistochemical staining of human AMD lesion. Histological sections of the outer eye wall were stained with the MDA-specific mAb MDA2 (top left panel) and a CFH-specific Ab (top right panel). Immunoreactivity of MDA is detected in all departments, whereas Factor H (CFH) distribution is restricted to drusen, Bruch's membrane and choroid. Epitopes recognized are shown in blue; bottom panels show respective control stains.

### DETAILED DESCRIPTION

The term “Factor H” or “complement Factor H” shall refer to the human Factor H protein or Factor H from other mammalian origin, e.g. murine, either from natural sources or recombinantly produced. The term includes polypeptide or peptide fragments, variants or derivatives of Factor H and Factor H related proteins that are capable of binding to the oxidation specific epitopes, MDA and/or MAA, thus, having the desired anti-MDA and/or anti-MAA activity. The relevant peptide fragments include the binding domains having specificity against MDA or MAA, respectively. For example, the following Factor H related peptides have proven to interact with MDA: peptides from complement Factor H related protein, Factor H precursor, putative Factor H-related protein B precursor and Factor H related protein C precursor, recombinantly expressed Factor H fragments. The term also refers to Factor H like protein 1 and Factor H related protein 1, 2, 3, 4 and 5

The term “oxidative stress” refers to an imbalance of the immune and/ or complement system caused by environmental or pathological factors, which triggers the development of oxidation specific epitopes.

The term “oxidation specific epitope” or “oxidation epitopes”, sometimes called “oxidized epitopes”, refers to neo-epitopes on structures, like lipids, lipoproteins, proteins or dead cells, including drusen and debris, which develop due to reactions caused by oxidative stress, in particular with oxidized lipoproteins. Apoptotic and necrotic cells are known to carry oxidation specific epitopes. Among those epitopes is MDA or epitopes of MAA adducted proteins, alone or associated with a carrier, e.g. a lipid or lipoprotein, such as LDL, or circulating microparticles, apoptotic and necrotic cells, cellular debris, or other epitopes formed by oxidation of endogenous structures.

The term “oxidative stress disease” refers to human or veterinary diseases, either chronic or acute, caused or associated with oxidative stress, including MDA- or MAA-induced inflammation and MDA- or MAA induced complement activation, thus e.g. triggering local or systemic inflammatory reactions. Among the oxidative stress diseases there is cardiovascular disease, the metabolic syndrome and obesity, autoimmune diseases, including rheumatoid arthritis, multiple sclerosis, cancer and conditions caused by cancer treatment, age related macular degeneration, Alzheimer's disease, brain senescence, alcoholic liver disease, ischemic reperfusion injury, diabetic nephropathy, nephritis, acute lung injury, and infectious diseases, or any inflammatory conditions associated or caused therewith. “Oxidative stress disease conditions” are generally understood as those pathological conditions associated with oxidative stress. Specifically the invention refers to the condition or disease that benefits from the inhibition of MDA and/or MAA.

The term “recombinant protein” refers to any polypeptide or protein that is encoded by a nucleic acid recombined in a host cell to produce said polypeptide or protein or a precursor thereof in a host cell culture.

The term “treatment” refers to both prophylactic and therapeutic treatment of patients. The term also includes treatment for in vivo or ex vivo diagnostic purposes. Treatment may be either human or veterinary, including treatment of mammalians in general. As used herein, and as well understood in the art, treatment is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. Treatment can also mean prolonging survival as compared to expected survival if not receiving treatment. Palliating a disease or disorder means that the extent and/or undesirable clinical manifestations of a disorder or a disease state are lessened and/or the time course of the progression is slowed or lengthened, as compared to not treating the disorder. The term prevention or prophylaxis, or synonym thereto, as used herein refers to a reduction in the risk or probability of a patient becoming afflicted with the disease or manifesting a symptom associated with the disease.

Thus, the invention refers to a new indication of Factor H, which is the prevention and/or treatment of oxidative stress disease conditions in a patient. It was surprisingly found out that Factor H acts as natural antagonist of oxidative stress processes and products, like MDA-LDL. Because of its binding to oxidation specific epitopes, like MDA and/or MAA, it is herein also called “MDA reactive protein”. Thereby it would inhibit complement activation, pro-inflammatory cytokine production and foam cell formation, and thus support the natural defense mechanism. This finding was unexpected since Factor H is a protein that is abundantly present in the circulation. It was believed that endogenous Factor H is not effectively inhibiting the alternative pathway of complement and thus believed not to be effective in treating ischemia/ reperfusion injury (sic Huan et al. above).

According to the invention it is surprisingly shown that Factor H has a significant impact on lipid peroxidation-derived products, such as MDA or MAA, that have been documented to play a role in various pathologic settings of oxidative stress.

Factor H was found to recognize structures derived from lipid peroxidation, which are present on oxidized LDL as well as on apoptotic cells. These epitopes represent danger signals and as such are recognized by several branches of the innate immune system. The phosphocholine-epitope for example is recognized by the natural IgM antibody T15 (Shaw et al. 2000 J Clin Invest 105: 1731), by the innate effector protein CRP as well as by the macrophage scavenger receptor CD36. The particular set of structures, which are the MDA modifications, is used as determinants in models of respective disease. MDA-modified proteins and lipids are present not only in atherosclerosis and on the membranes of apoptotic cells, but accumulate in various inflammatory settings ranging from acute lung injury to Alzheimer's disease and rheumatoid arthritis. Using the ubiquitous MDA-epitope as a model, it was surprisingly found that Factor H is the natural binder of such products of oxidative stress, thereby supporting the innate immune defence in such settings.

In respective experiments MDA-modified carriers were used to pull down potential candidates from mouse serum. Surprisingly, the vast majority of the peptides found exclusively in the MDA-pulldown were attributed to one single protein, which turned out to be complement Factor H. Results were confirmed by Western Blotting with a Factor H-specific antibody. Factor H was exclusively detected in the MDA-pulldown both in mouse and human plasma.

The interaction of Factor H with MDA was confirmed by ELISA, proving that Factor H bound to MDA in a specific and calcium-independent manner. Factor H bound only to MDA-modified LDL but not to native LDL, and this was independent of the protein backbone. For example, Factor H also bound to MDA-modified BSA but not to unmodified BSA. The specificity of the binding was checked using a competition assay. It was found that only MDA-modified LDL competed for the binding of MDA-LDL to coated Factor H, whereas unmodified native LDL and CuOx-LDL (CuSO₄ oxidized LDL), which carries oxidative modifications of a different kind, had no impact at all.

Upon confirming the specificity of this binding, the region of Factor H was determined, which was important for the binding to MDA. Therefore, the interaction of MDA with recombinant fragments of Factor H was studied employing respective immunoassays. MDA binding has proven when domain 7 (CCR7) or 20 (CCR20) or both were present.

It was found that Factor H may play a direct protective role in AMD. A study employed sections of the outer eye wall from AMD cases to perform immunohistochemistry. Both MDA as well as Factor H were present in the lesion. It could be shown for the first time that on necrotic retinal pigment epithelium cells, Factor H binding colocalizes completely with the MDA-epitopes. Importantly, this binding could be competed with MDA-modified protein, indicating that in fact MDA is the main binding partner for Factor H on the surface of dead cells. This interaction between Factor H and MDA has therefore proven to play a major role in the pathogenesis of age related macular degeneration.

It could also be shown that Factor H was able to block MDA-induced inflammation. THP1 macrophages were treated with MDA-BSA, which induces the expression of a number of cytokines, as a result of inflammatory processes, most prominently IL8 (interleukin 8) and MIP2 (macrophage inflammatory protein 2). Importantly, addition of Factor H almost completely blocked this effect. This was validated also on the protein level.

The complement inhibitory activity of Factor H has also proven. In fact, it was demonstrated, that addition of MDA-BSA to human serum directly activated complement. This effect would be inhibited by binding of Factor H.

Thus, in analogy to the binding of CRP to PC, Factor H is used according to the invention as an MDA reactive protein binding to the MDA-epitope, thereby supporting the innate defense mechanism against the consequences of oxidative stress. This is of importance in many acute or chronic inflammatory settings including AMD.

Since oxidative stress is directly linked to the development of AMD, it can be shown that the Factor H polymorphism has also an impact on the binding ability of Factor H to MDA, which is a major product of oxidative stress. Importantly, it was found that the Factor H Y402H variant displays severely impaired binding to MDA.

The potency of the endogenous Factor H to bind MDA and/or MAA, either determined by the amount of functional Factor H or specific Factor H activity present in a sample, would be a direct measure of the protection of a patient and the prognosis of oxidative stress disease. This is of particular importance in patients suffering from increased risk factors of oxidative stress disease, such as an increased level of LDL and/or low or dysfunctional level of HDL, or an increased TBARS level. Those patients, who might already have a diagnosis of an early disease stage, might benefit from the determination or monitoring of the Factor H potency, which would protect against the outbreak or development of disease. If the endogenous Factor H would be reduced or less functional than the Factor H in normal blood plasma, treatment of disease associated with such acquired Factor H deficiency, by a suitable Factor H preparation or other measures would be indicated. The treatment is specifically indicated, if the Factor H level or activity is reduced by at least 20%, preferably by at least 30%, 40% or 50%.

The protective effect of endogenous Factor H is preferably determined by a potency assay, e.g. a binding assay to determine the anti-MDA or anti-MAA activity of Factor H, or a functional MDA or MAA assay, wherein the effects of MDA or MAA and the respective physiological impact of MDA or MAA to physiological processes, like complement activation or interleukin release, is inhibited by Factor H. Thereby a potency assay of Factor H binding to MDA or MAA can be provided based on an immunoassay. For example, a preferred binding at least 1 mg, preferably at least 2 mg, 3 mg, 5 mg, 10 mg or even more MDA-modified protein (such as MDA-LDL with 75% of lysines modified, i.e. 270 lysines modified per molecule) per gram Factor H is determined in such potency assay. MDA-modified protein is coated at a limiting concentration on a protein binding plate and incubated with a limiting concentration of Factor H. The amount of bound Factor H can be measured using a Factor H specific antibody.

By means of such an assay endogenous Factor H can be determined as a biomarker to assess the risk for developing oxidative stress disease, in particular cardiovascular disease, which specifically provides for an atheroprotective effect.

On the other hand the MDA and/or MAA structures can be assessed as biomarkers of oxidative stress disease employing Factor H as reactand. The relevant inflammatory settings are usually associated with an increased level of an oxidative stress marker and may be determined employing respective assays. According to the invention there is preferably provided a new assay for determining the marker MDA or MAA adducted proteins based on binding of the marker to Factor H. The exemplary immunoassay employs the immobilization of circulating Factor H via a specific capturing antibody, followed by the detection of bound MDA-modified proteins using MDA-specific antibodies.

The appropriate saturation binding assay typically would provide for the determination of the binding potency, affinity and/or avidity.

The present invention includes treatment with a pharmaceutical preparation, containing as active substance Factor H in a therapeutically effective amount, specifically functional Factor H with anti-MDA activity, optionally in combination with pharmaceutically acceptable conventional excipients and/or carriers. In particular a pharmaceutically acceptable formulation of Factor H is compatible with the treatment of a patient, including all members of the animal kingdom, especially mammals, including human. The subject or patient is suitably a human.

The term therapeutically effective amount, effective amount or sufficient amount of Factor H is a quantity or activity sufficient to, when administered to the subject effect beneficial or desired results, including clinical results, and, as such, an effective amount or synonym thereof depends upon the context in which it is being applied. For example, in the context of MDA inhibition, it is an amount of the compound sufficient to achieve an inhibition of MDA effects compared to the response obtained without administration of the compound. In the context of disease, therapeutically effective amounts of Factor H are used to treat, modulate, attenuate, reverse, or affect a disease or conditions that benefits from an inhibition of MDA, for example, acute or chronic inflammatory diseases. An effective amount is intended to mean that amount of a compound that is sufficient to treat, prevent or inhibit such diseases or conditions. The amount of Factor H that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art. Also, as used herein, a therapeutically effective amount of Factor H is an amount which prevents, inhibits, suppresses or reduces a disease or conditions that benefits from an inhibition of MDA, for example, chronic inflammatory diseases as determined by clinical symptoms in a subject as compared to a control. As defined herein, a therapeutically effective amount of Factor H may be readily determined by one of ordinary skill by routine methods known in the art.

Moreover, a treatment or prevention regime of a subject with a therapeutically effective amount of Factor H may consist of a single administration, or alternatively comprise a series of applications. For example, Factor H may be administered at least once a month. However, in another embodiment, Factor H may be administered to the subject from about one time per week to about a daily administration for a given treatment. The length of the treatment period depends on a variety of factors, such as the severity of the disease, the age of the patient, the concentration and the activity of Factor H. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

Examplary formulations as used for parenteral administration include subcutaneous, intramuscular, intravenous or intraocular injection as, for example, a sterile solution or suspension. Exemplary formulations for topical or mucosal administration including intranasal, intrabronchial or intraocular use include aqueous or oily suspensions or solutions, emulsions, eventually used as sprays, drops or ointment.

Determination of a therapeutically effective amount or a prophylactically effective amount of Factor H according to the invention can be readily made by one skilled in the art, by the use of known techniques and by observing results obtained under analogous circumstances. In vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a skilled artisan.

A parenteral or mucosal dose of Factor H to increase the endogenous level to the normal range is preferred. It is preferred that a loading dose, e.g. by bolus infusion, of 30-100 IU/kg, is followed by a maintenance dose of 5-50 IU/kg over several days. One Factor H unit (IU) corresponds to the quantity of functional Factor H in 1 ml in pooled normal human plasma. Administration of 1 IU Factor H per kg body weight increases the Factor H concentration (activity) by approximately 1%

The dose requirement is calculated according to the following formula:

Number of units (dose)=body weight (kg)×(100−current Factor H activity (in %)).

Initially, a Factor H level of at least 80%, preferably at least 100% should be achieved, and this should be kept beyond 50%, preferably more than 70% or 90%, during the treatment. The dose should be defined after determination of the patient's Factor H activity.

The preferred Factor H preparation as used according to the invention would have a reduced content of any dysfunctional Factor H variants, such as those associated with decreased MDA- or MAA-binding, e.g. the Y402H mutations, or exclude such variants.

Those patients with Factor H variants associated with decreased MDA- or MAA-binding, such as Y402H Factor H polymorphism are considered high risk patients of oxidative stress diseases, and would be considered as preferred patients for treatment with Factor H.

The further preferred Factor H preparations are purified from any contaminating structures bearing the MDA and/or MAA epitopes. Since plasma Factor H is obtained in a plasma fraction containg both, Factor H and complexes of Factor H with MDA and/or MAA, it is desired to separate the contaminating immune complexes and MAA/MAA structures from the active ingredient Factor H. Factor H like other plasma derivatives is therefore preferably obtained by purification employing affinity chromatography using ligands binding to MDA and/or MAA epitopes, such as peptide ligands, antibodies, antibody fragments or derivatives, optionally bound to a chromatographic material. The plasma derivative in the eluate is thereby purified from the MDA and/or MAA structures. Thereby undesired side effects of plasma derivatives caused by those structures can be avoided.

The present invention is further illustrated by the following examples without being limited thereto.

### EXAMPLES

### Example 1

### Preparation of MAA-Modified Proteins

MAA-modified proteins were prepared by incubating them at a concentration of 2 mg/ml for 3 hours at 37° C. with 100 mM Malondialdehyde (MDA) and 200 mM Acetaldehyde in PBS pH 4.8. MDA (0.5 M) was freshly generated from 1,1,3,3 Tetramethoxypropane by acid hydrolysis: 1,1,3,3 Tetramethoxypropane was incubated with 12 μl 4 N HCl and 400 μl H₂O at 37° C. for 10 min. The reaction was stopped by adjusting the pH to 4.8 by addition of 1 N NaOH, and the volume was brought to 1 ml with H₂O. After conjugation, MAA-adducted protein was extensively dialyzed against PBS to remove any unreacted MDA.

### Example 2

### Preparation of MAA-Polylysine Coated Beads

Unmodified or MAA-modified polylysine (generated as described above from polylysine 1.4 kD SigmaAldrich) was coupled to NHS-activated sepharose (GE Healthcare) according to manufacturer's instructions.

### Example 3

### Pulldown Procedure for MDA-Binding Plasma Proteins

Plasma obtained from LDLR-/-RAG-/-mice or human donors were diluted to a concentration of 1 mg/ml total protein. To minimize the amount of proteins binding to unmodified polylysine, plasma dilutions were incubated with PL-coupled beads for 2 h at 4° C. After the incubation, supernatant was incubated with either polylysine- or MAA-polylysine-beads for another 2 h at 4° C. Beads were washed 3 times with TBS (pH 7.4, 500 mM NaCl, 0.5% NP−40, 2 mM CaCl₂, 1 mM MgCl₂). After the last wash, bound proteins were dissociated by adding LDS-sample buffer (Invitrogen) and heating at 95° C. for 5 min. The supernatants were separated by SDS-PAGE and analyzed by LC-MS-MS on a quadrupole time-of-flight (QTOF) mass spectrometer (Waters) coupled to a nano HPLC system (Agilent). Obtained data was searched against IPI_MOUSE database v.3.32 appended with known contaminants (e.g. human keratin proteins).

### Example 4

### Verification of the Interaction with Immunoblotting

The samples were separated by SDS-PAGE and blotted on a PVDF-membrane. The membrane was blocked in 5% non-fat dry milk in PBS with 0.05% Tween 20 and probed for Factor H with goat polyclonal anti-human-Factor H (Calbiochem, 1:10000 in antibody diluent (1% non-fat dry milk in PBS with 0.05% Tween 20)) or goat polyclonal anti-mouse-Factor H (SantaCruz, 1:1000 in antibody diluent). Anti-goat-IgG coupled to horseradish peroxidase (Calbiochem, 1:5000 in antibody diluent) was used as a secondary antibody. Factor H was exclusively detected in the pulldown with MAA-polylysine, but not in the pulldown with unmodified polylysine.

### Example 5

### Verification of the Interaction with Enzyme Linked Immunosorbent Assay

MAA-LDL and MAA-BSA at a concentration of 5 μg/ml in PBS containing 0.27 mM EDTA were added to each well of a 96-well white, roundbottomed microtitration plate (Thermo, MicrofluorII roundbottom) and incubated 1 h at 37° C. After washing and blocking steps, the plate was incubated with purified Factor H (Calbiochem) at a concentration of 1-5 μg/ml in TBS-BSA (TBS pH 7.4, containing 1% BSA) overnight at 4° C. Bound Factor H was detected with goat polyclonal anti-human-Factor H (Calbiochem, 1:10000 in TBS-BSA) and mouse anti-goat-IgG coupled to alkaline phosphatase (SigmaAldrich, 1:30000 in TBS-BSA). Lumiphos (Lumigen, 50% solution in water) was used as substrate and the luminescence signals were measured on Victor Luminometer (Wallac II, Perkin Elmer) or BioTek and results expressed as relative light units (RLU) per 100 ms. Factor H clearly bound only to MAA-modified, but not to native proteins with at least 100-fold stronger signals.

### Example 6

### Competition Assay to Assess the Specificity of the Interaction

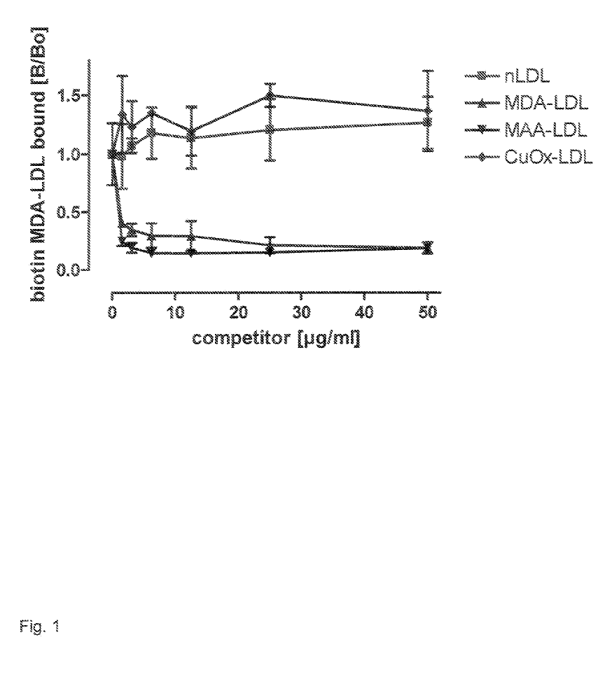

To check the specificity of this binding, we developed a competition assay: Purified Factor H (Calbiochem) or MAA-BSA were coated at a concentration of 2 μg/ml or 0.5 μg/ml, respectively. Biotin-labeled MDA-LDL or Factor H at a concentration of 0.5 μg/ml or 1 μg/ml (in TBS-BSA) were mixed with indicated concentrations of unlabeled native LDL, MDA-LDL, MAA-LDL or oxidized LDL and plated o/n at 4° C. Bound biotin-MDA-LDL was detected with NeutrAvidin coupled to alkaline phosphatase (PerkinElmer, 1:10000 in TBS-BSA). Bound Factor H was detected with goat polyclonal anti-human-Factor H (Calbiochem, 1:10000 in TBS-BSA) and mouse anti-goat-IgG coupled to alkaline phosphatase (SigmaAldrich, 1:30000 in TBS-BSA). Substrate was added and luminescence was measured as described above. As seen in FIG. 1, only MDA or MAA—modified LDL could compete for the binding, whereas unmodified or CuSO₄ oxidized LDL did not compete.

### Example 7

### Co-Factor Assay to Assess the Functional Relevance of MDA-Bound Factor H.

An important complement regulatory activity of CFH lies within its capacity to act as a cofactor for the serine protease factor I, thereby promoting the degradation of C3b into inactive iC3b fragments. This is also important, as the deposition of iC3b on apoptotic cells increases their clearance in an anti-inflammatory manner. We therefore tested whether CFH induces iC3b generation when bound to immobilized MDA.

MAA-BSA at a concentration of 5 μg/ml in PBS was bound to the surface of a 96-well flat-bottom microtitration plate (NUNC Maxisorp). After washing and blocking, Factor H was added to coated MAA-BSA at concentrations of 0.2-5 μg/ml in PBS-BSA (PBS pH 7.4, containing 1% BSA) for 1 h at room temperature. Unbound protein was removed by washing and plates were incubated with C3b (Comptech, 0.8 μg/ml) and factor I (Comptech, 0.2 μg/ml) in PBS for 90 min at 37° C. The reaction was stopped by adding LDS-sample buffer (Invitrogen) and samples were denatured at 95° C. for 5 min.). The generation of iC3b fragments was assessed by SDS-PAGE followed by immunoblotting. MAA-bound Factor H resulted had the capacity to induce Factor I dependent iC3b generation.

### Example 8

### Mapping of the Binding Domain Using Recombinantly Expressed Factor H Fragments

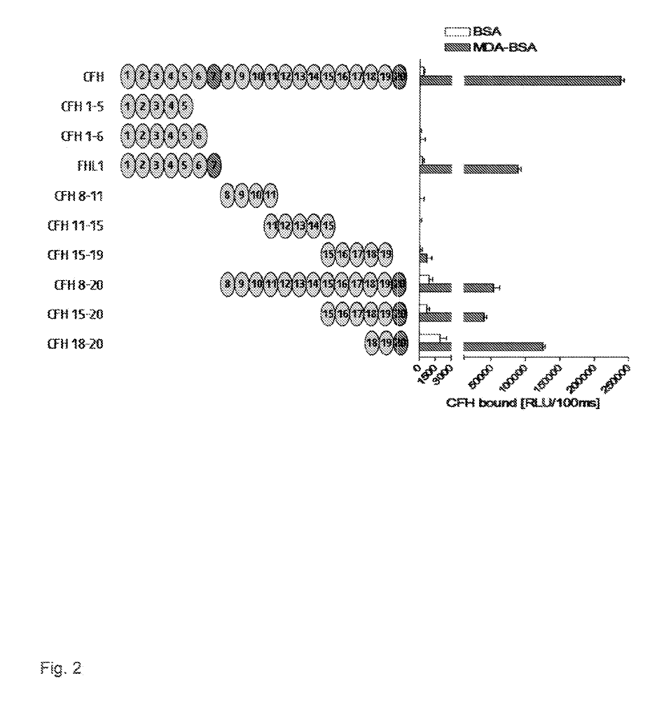

Recombinantly expressed FHL1 as well as Factor H fragments were prepared in the pBSV-8His baculovirus expression system. Shortly, Spodoptera frugiperda (Sf9) cells were grown in expression medium (BioWhittaker, Verviers, Belgium) supplemented with streptomycin (100 μg/ml), penicillin (100 U/ml), and amphotericin B (250 ng/ml) and infected with a recombinant baculovirus at a multiplicity of infection of 5. The culture supernatant was collected 9 days after infection, and recombinant proteins were purified by Ni⁺-chelate chromatography or by Äkta fast-performance liquid chromatography purification (Pharmacia, Piscataway, N.J.). The proteins were concentrated using Centricon microconcentrators with a cutoff at 10 kDa (Millipore, Bedford, Mass.). The constructs were coated at a concentration of 5 μg/ml and incubated with biotinylated BSA or MAA-BSA at 5 μg/ml. Bound biotin was detected with NeutrAvidin as described above. FIG. 2 shows that only if complement control region 7 or 20 or both are present in the construct, MAA-BSA can bind.

### Example 9

### Comparison of MDA-Binding Capacity of Factor H Variants 402Y and 402H

To compare the MAA-binding of the Factor H variants 402Y and 402H, MAA-BSA was coated at a concentration of 1 μg/ml and incubated with both variants at a concentration of 1 μg/ml. Bound Factor H was detected with the anti-Factor H antibody mentioned above. At the indicated concentrations, the 402H variant showed more than 55% decreased binding compared to Factor H 402Y.

### Example 10

### Immunohistochemical Staining of Human Eye Sections

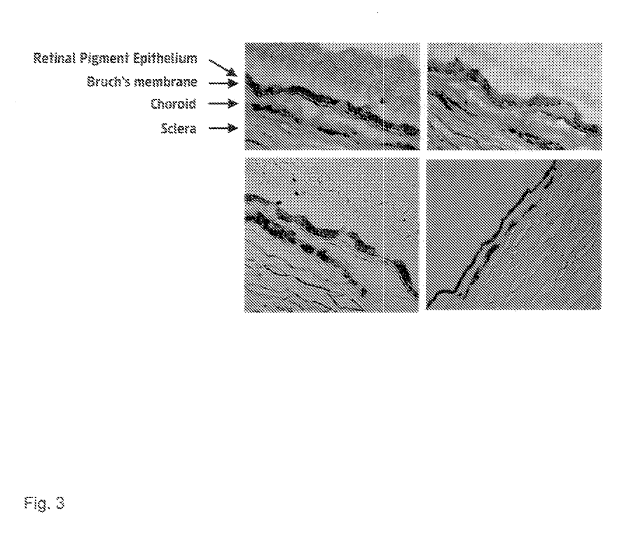

Histological sections of the outer eye wall of a 98 y/o AMD-patient were stained with antibodies directed against MDA (mouse monoclonal antibody MDA2) and Factor H (guinea pig antiserum reacting with human Factor H). Both MDA as well as Factor H were present in the lesion area (FIG. 3).

### Example 11

### Neutralization of MAA-Mediated Cytokine Secretion with Factor H

Human THP-1 monocytic cells were cultured in RPMI-1640 supplemented with 10% FCS. The stimulation medium contained BSA or MAA-BSA at 50 μg/ml and/or Factor H at 200-12.5 μg/ml and was incubated for 30 min at RT before plating. Before stimulation, cells were washed with serum-free RPMI-1640 and incubated with the stimulation medium at a density of 5×10⁵ cells/ml for 14 h. Cells were removed by centrifugation (500 g, 10 min) and supernatants stored at −80° C. The supernatants were assayed for the presence of IL8 with a commercially available ELISA-Kit (OptEia Human IL8 ELISA Set, BecktonDickinson) according to manufacturer's instructions. Treatment with MAA-BSA raised the levels of IL8 from about 150 pg/ml to about 1650 pg/ml. Coincubation with Factor H at 200 μg reduced the amount of secreted IL8 to about 500 pg/ml.

### Example 12

### Complement Activation in Human Sera with MAA-BSA

Human serum was collected from healthy volunteers in serum tubes (Vacuette 8 ml Z Serum Sep Clot Activator) using a 21 G needle (Vacuette blood collection set+luer adapter). After coagulation, the thrombus was removed by centrifugation (15 min 2000 g 4° C.), serum was aliquotized and stored at −80° C. For complement activation, one volume of serum was mixed with one volume of a solution of BSA/MAA-BSA in Veronal Buffered Saline (1 mg/ml or less), followed by an incubation for 20 min at 37° C. As a positive control, serum was mixed with Cobra Venom Factor in VBS (20 U/ml). The complement reaction was stopped by adding 3 volumes of cold sample buffer (Quidel) and C3a generation was determined with Microvue quantitative C3a ELISA (Quidel). Addition of MAA-BSA at a final concentration of 500 μg/ml resulted in a 2-fold increased C3a-generation compared to incubation with buffer or native BSA.

1-16. (canceled)

17. A method of determining the specific binding of Factor H to malondialdehyde (MDA) and/or malondialdehyde-acetaldehyde (MAA) in a sample, comprising:
providing a reactand selected from the group consisting of Factor H, a MDA epitope and a MAA epitope;
incubating the reactand with a sample of body tissue or body fluid; and
determining reaction products resulting from a reaction of the reactand with the sample.

18. The method of claim 17, wherein endogenous Factor H is determined in the sample as a biomarker to assess the risk of developing oxidative stress disease or as a prognostic factor of oxidative stress disease.

19. The method of claim 17, further comprising the step of using MDA binding or MAA binding to determine Factor H potency to protect against the outbreak or development of oxidative stress disease.

20. The method of claim 17, wherein an acquired Factor H deficiency is determined by reduced MDA binding or MAA binding.

21. The method of claim 17, wherein a reduced serum MDA binding capacity of serum Factor H is determined, the reduced serum MDA binding capacity being at least 25% less than in normal human serum.

22. The method of claim 21, wherein the reduced serum MDA binding capacity is associated with the 402H Factor H variant.

23. The method of claim 17, wherein MDA and/or MAA structures are determined in the sample.

24. The method of claim 17, wherein an immunoassay is employed, the immunoassay comprising MDA and/or MAA adducted proteins which specifically bind to Factor H.

25. The method of claim 24, wherein a saturation binding assay is employed.

26. The method of claim 17, wherein wherein an immunoassay is employed, the immunoassay comprising a Factor H specific antibody or MDA-specific antibody.

27. The method of claim 24, wherein a saturation binding assay is employed.

28. The method of claim 17, wherein a functional MDA assay or a functional MAA assay is employed.

29. The method of claim 17, wherein the sample is selected from the group consisting of blood, plasma or serum.

30. The method of claim 17, wherein the sample is from a patient suffering from increased risk factors of oxidative stress disease.

31. The method of claim 30, wherein the patient has a diagnosis of an early stage disease.

32. The method of claim 30, wherein the oxidative stress is associated with a medical condition selected from the group consisting of cardiovascular disease, metabolic syndrome, obesity, an autoimmune disease, multiple sclerosis, cancer and conditions caused by cancer treatment, age related macular degeneration, Alzheimer's disease, brain senescence, alcoholic liver disease, ischemic reperfusion injury, diabetic nephropathy, nephritis, acute lung injury, an infectious disease, and an inflammatory condition associated with or caused by one of the foregoing.

33. The method of claims 30, wherein the patient suffers from a disease condition associated with an increased level of at least one oxidative stress marker selected from the group consisting of oxidized lipoproteins, oxidized lipids, oxidized proteins, circulating microparticles, necrotic cells, apoptotic cells, cellular debris, Factor H complexes, and an increased level of thiobarbituric acid reacting substances (TBARS).

34. The method of claim 17, wherein the reactand is bound to a solid surface.
